(19) United States
(12) Patent Application Publication
(10) Pub. No.: US 2017/0143303 A1
(43) Pub. Date: May 25, 2017

(54) AUTOMATED ULTRASOUND KNEE SCANNER

(71) Applicant: GENERAL ELECTRIC COMPANY, Schenectady, NY (US)

(72) Inventors: Dongqing Chen, New Berlin, WI (US); Menachem Halmann, Waukesha, WI (US); Jeffrey Scott Peiffer, Delafield, WI (US); Craig Robert Loomis, Shorewood, WI (US); Eunji Kang, South Lyon, MI (US)

(73) Assignee: GENERAL ELECTRIC COMPANY, Schenectady, NY (US)

(21) Appl. No.: 14/947,670

(22) Filed: Nov. 20, 2015

(51) Int. Cl. A61B 8/00; A61B 34/32; A61B 8/08; A61B 34/10

(52) U.S. Cl. CPC A61B 8/4218; A61B 34/10; A61B 34/32; A61B 8/0875; A61B 2034/2055

(57) ABSTRACT

A method of acquiring ultrasound of a patient's knee includes obtaining a digital picture of a patient's knee with a digital camera. The method further includes applying instructions to the processor to identify the position of the patient's knee relative to a known datum based on an algorithm and the digital picture. A scan path is created about the patient's knee based on the position of the patient's knee. Instructions are provided from a robotic processor to robotically move an ultrasound probe along the scan path.

### CROSS-REFERENCE TO RELATED PATENT APPLICATIONS

None

### BACKGROUND

The present invention relates generally to the field of ultrasound and more particularly, to automated ultrasound for diagnosis and monitoring of knee rheumatoid arthritis and knee osteoarthritis.

Rheumatoid arthritis is an autoimmune disease in which the body attacks itself. The body attacks the soft lining around the joints including the synovium membrane. The synovium membrane is found in all musculoskeletal joints, such as: the knee joint and shoulder joint. The thin synovium membrane surrounds the inner lining of the knee joint and may have folds and/or fringes. A function of the synovium membrane is to create synovial fluid, which helps lubricate the joint. Rheumatoid arthritis results in fluid buildup around the joint causing the knee joint to be tender, warm, swollen and can be severely painful.

Osteoarthritis is a degenerative joint disease in which a person experiences a breakdown of the cartilage that cushions the joints. The wearing down of cartilage causes the bones to rub against each other, which accounts for inflammation and pain.

### SUMMARY

In one embodiment an apparatus includes a digital camera providing a digital picture of a patient's knee. A processor receives instructions to identify the position of the patient's knee from the digital picture. The processor creates a scan path about the patient's knee. A robotic arm supports an ultrasound probe and receives instructions from the processor to move the ultrasound probe along the scan path.

In one embodiment a method includes obtaining a digital picture of a patient's knee with a digital camera and applying instructions to the processor to identify the position of the patient's knee relative to a known datum based on an algorithm and the digital picture. The method further includes creating a scan path about the patient's knee based on the position of the patient's knee and providing instructions from a robotic processor to robotically move an ultrasound probe along the scan path.

### BRIEF DESCRIPTION OF THE DRAWINGS

FIG. 1 is a schematic view of an automated ultrasound system.

FIG. 2 is a schematic view of a patient support.

FIG. 3 is a schematic side view of the patient support of FIG. 2 in a second position.

FIG. 4 is an isometric view of a patient's leg including the patient's knee.

FIG. 5 is a schematic view of a robotic arm.

FIG. 6 is a flow diagram of an example method for acquiring ultrasound images of a patient's knee.

FIG. 7 is a schematic view of a scan path.

### DETAILED DESCRIPTION OF EXAMPLES

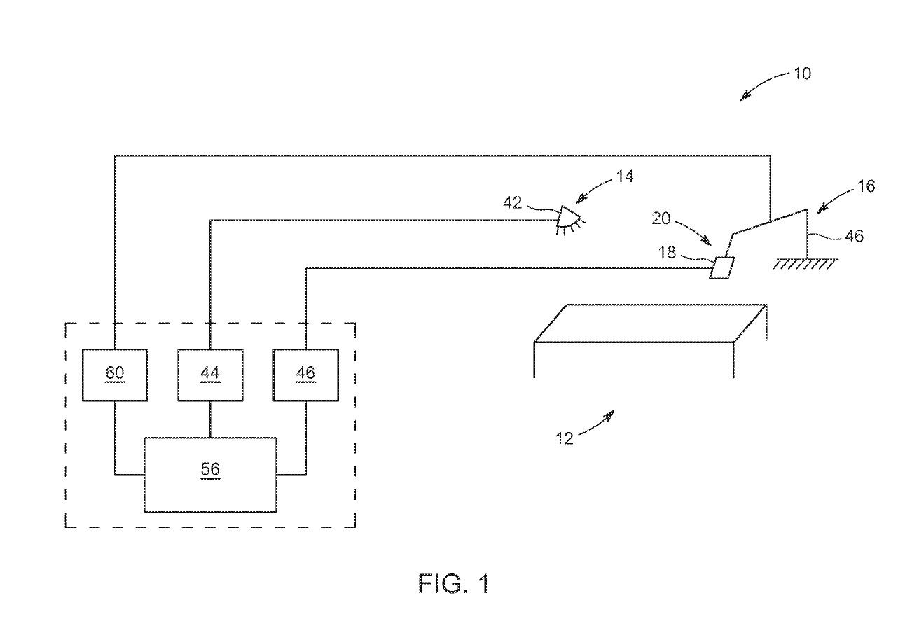

Referring to FIG. 1, an automated ultrasound system 10 includes a patient support 12 configured to position a patient's knee; an imaging system 14 to capture an image of an outer portion of a patient's leg about the patient's knee; a robotic system 16 configured to move an ultrasound probe 18 of ultrasound system 20 about the outer portion of a patient's leg; and a control system 22 operatively controlling and/or receiving data from one or more of the imaging systems 14, robotic system 16 and the ultrasound system 20.

As used herein ultrasound imaging includes but is not limited to two dimensional ultrasound, three dimensional volumetric ultrasound imaging, and four dimensional volumetric ultrasound imaging. Where two dimensional and three dimensional ultrasound imaging includes two and three spatial dimensions respectively. Alternatively, two dimensional ultrasound may include one spatial and one temporal dimension and three dimensional ultrasound imaging may include two spatial dimensions and one temporal dimension. Four dimensional ultrasound includes three spatial dimensions and one temporal dimension.

In one implementation three dimensional ultrasound imaging converts standard two dimensional spatial ultrasound images into a volumetric data set. The three dimensional image is then used to analyze the patient's joints including but not limited to the patient's knee to assist in the diagnosis of a patient's medical condition.

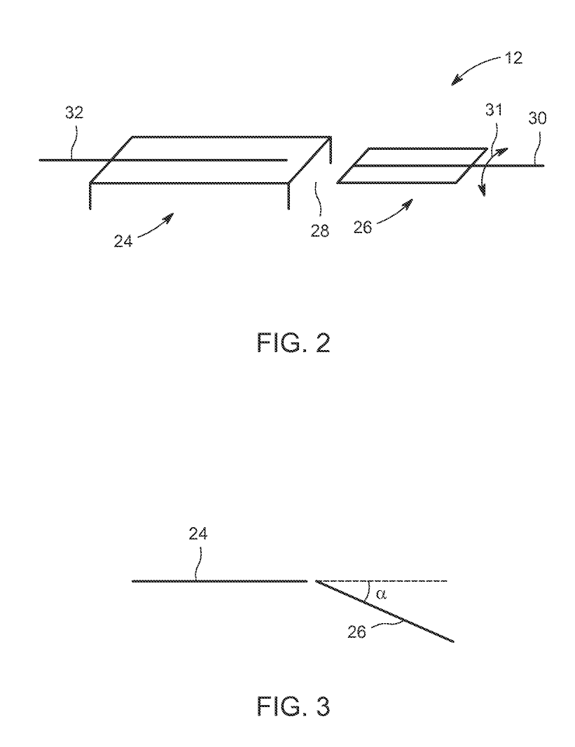

Referring to FIG. 2, in one implementation the patient support 12 includes a bed 24 having an extension portion 26 which supports a patient's leg such that the patient's knee is in a horizontal position the patient's leg extends in a general vector direction normal to the direction of gravity. In another implementation patient support 12 includes a chair portion and an extension portion extending in a horizontal orientation. In one implementation the horizontal orientation is perpendicular to the direction of gravity.

It is also contemplated that the bed 24 and extension portion 26 may be positioned in an orientation other than generally horizontal such that a patient's leg bends at the knee joint 34. In one implementation the bed 24 and/or extension portion 26 may include a gap 28 such that the patient's knee may be viewed 360 degrees about the knee joint 34. Stated another way extension portion 26 may include a gap 28 either within the extension portion 26 or between extension portion 26 and the bed 24 to provide access for the ultrasound probe 18 to be placed or proximal proximate to the outside of a patient's leg about the entire outer periphery or skin 36 of the patient's leg proximate and at the knee joint 34. Ultrasound probe 18 may be positioned by the robotic system 16 about the patient's leg adjacent the knee joint 34. In this manner the ultrasound probe may contact the outer portion or the skin 36 of a patient's leg to obtain an ultrasound scan of the knee joint 34. In one implementation the ultrasound probe need not directly contact the patient's skin but rather there is coupling media between the ultrasound probe and the patient's skin. In one embodiment, there may be another transmit media as known in the art such as water or other known media. In the implementation in which contact with the patient's skin is not required the scan path 70 as discussed herein below is determined by an algorithm as a function of the location of the patient's leg. The outer portion of the patient's knee includes, but is not limited to the region proximate to the patella 38, popliteal fossa 40 (knee pit) and the regions of the leg and knee joint between the patella and the popliteal foss. Access to all areas of the patient's leg allows the system 10 to obtain an ultrasound scan of all of the anatomical portions of the knee joint.

Referring to FIG. 3 the extension portion 26 may be positioned such that a longitudinal axis 30 of extension portion 26 forms a non-collinear angle 25 with a longitudinal axis 32 of the bed 24. In one implementation extension portion 26 includes two parts that are movable relative to one another, wherein each part has a longitudinal axis that is not collinear with each other. In one implementation the two portions of extension portion 26 may be moved such that the longitudinal axis of each portion is moved from a non-collinear orientation to a co-linear orientation. In a further implementation, the two portions of extension portion 26 or bed 24 and extension portion 26 may rotate about a respective longitudinal axis 30 in a direction 31 to provide rotation of the patient's knee to place the knee joint 34 in different orientations for purposes of ultrasound examination.

In one implementation extension portion 26 is robotically controlled by a patient support processor (not shown) to provide instructions to motors operationally connected to the patient support to move the extension portion 26 relative to bed 24 based on instructions to reposition the patient's knee as described herein below. The patient support processor may be part of the processor 56 of controller 22.

The imaging system 14 includes a camera 42 operatively positioned to obtain an image of an outer portion 36 of a patent's knee. In one implementation the camera 42 is a digital camera capturing digital pictures that are sent to an image processor 44 having or receiving instructions to process the digital pictures to create a three dimensional model of the surface of the outer portion of the patient's leg, including the patient's knee. In one implementation the camera 42 includes more than one spaced digital camera. In one implementation the digital camera 42 takes more than one digital picture. In one implementation the digital camera 42 may be located in a fixed location relative to patient support 12. Where there is more than one camera, the cameras may be positioned from one another in a fixed location relative to the patient support 12. In one implementation the digital camera 42 may be secured to a portion of the robotic system 16 and the camera may be moved about a patient's knee by the robotic system 16. In one implementation the imaging system 14 may include a laser scanner or other image acquisition systems known in the art to obtain three-dimensional images of the patient's leg including the patient's knee.

The outer portion 36 of a patient's knee is defined herein as the exposed surface of the patient's skin in the area of the patient's knee joint. In one implementation the imaging system 14 includes a plurality of cameras 42 positioned to capture digital pictures of the outer portion 36 of a patient's knee sufficient to create a three dimensional model of the outer portion 36 of the patient's knee.

The images captured by the digital camera 42 are transmitted to the image processing controller to process the image data and through an algorithm to create a three dimensional model of at least an outer portion of the patient's knee. The three dimensional model includes a location relative to the robotic assembly system for robotic control and movement of the ultrasound probe 18 relative to at least part of the outer portion of the patient's knee.

In one implementation the acquisition of the image from a camera 42 and movement of a robotic arm 46 of the robotic system 16 and the ultrasound probe 18 occurs serially. First, the camera 42 acquires the image of the outer portion of the knee, then based on the acquired knee image, the system automatically detects or determines the Region of Interest (e.g. the pathology area). Then the system drives the robotic arm and the ultrasound probe to the region of interest (ROI) and starts the scan.] As the first position is determined for contact with the ultrasound probe 18 to a position on the outer portion of the patient's knee, a second location is determined based on an image of the location of the ultrasound probe and the patient's knee. As described herein below a scan path 70 of the ultrasound probe 18 may be updated as the ultrasound probe is moved about the patient's knee. The updating of the scan path 70 minimizes error in the calculated movement of the robotic arm and the ultrasound probe as it takes into account any movement of the patient's knee from a first time in which a first digital picture is obtained by the imaging system 14 to a second later time.

The image processing system 14 includes a processor or processing unit 44 that receives the images of the outer portion 36 of the patient's leg and knee from an optical device such as a scanner or a camera or directly through digital processing. For purposes of this application, the term “processing unit” shall mean a presently developed or future developed processing unit that executes sequences of instructions contained in a memory. In one example the term “memory” as used herein comprises a non-transient computer-readable medium containing computer code for the direction of controller. Execution of the sequences of instructions causes the processing unit comprising controller to perform steps such processing and storing the digital signals received from the cameras or other vision devices. The instructions may be loaded in a random access memory (RAM) for execution by the processing unit from a read only memory (ROM), a mass storage device, or some other persistent storage. In other implementations, hard wired circuitry may be used in place of or in combination with software instructions to implement the functions described. For example, the processing unit 44 may be embodied as part of one or more application-specific integrated circuits (ASICs). Unless otherwise specifically noted, a processing unit as used herein is not limited to any specific combination of hardware circuitry and software, nor to any particular source for the instructions to be executed.

The images are processed by the image processor 44 with an algorithm to create a three dimensional mapping of the outer portion of the patient's knee with a plurality of points where each point of the patient's knee has a three dimensional reference such as a Cartesian, x, y and z values or a spherical coordinate system.

The control system 22 includes a control processor 56 that generates a scan path 70 based on an algorithm that is a function of general knee geometry and anatomy and the three dimensional mapping of the outer portion of the patient's knee. The scan path 70 in one implementation is a plurality of discrete points that are used to generate a scan path 70 and the robotic arm will be instructed to follow the scan path 70 to provide an ultrasound scan of the patient's knee. The control processor 56 provides instructions to the robotic system 16 to drive the robotic arm 46 such that the interface 48 of the ultrasound probe 18 that is operatively secured to an end effector of the robotic arm positioned about the patient's knee to obtain an ultrasound scan of the patient's knee.

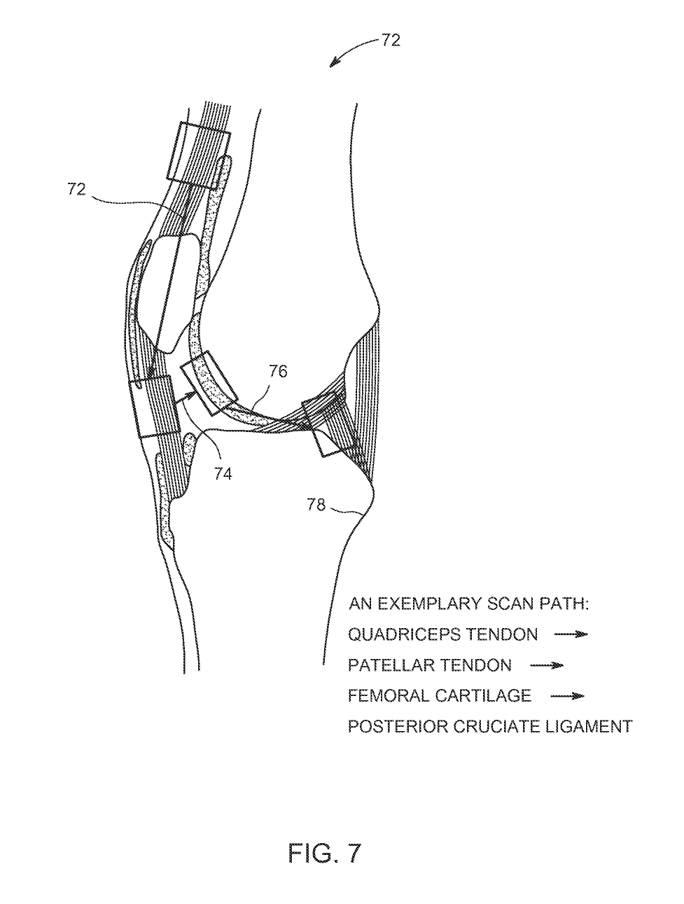

Referring to FIG. 7 an exemplary scan path 70 is illustrated. The scan path 70 may include one continuous path or multiple paths each with discrete start and stop points. In one implementation the scan path 70 includes a first path 72 that extends from a position above the patella, over the Patella and below the patella. As referred to herein the term above as used in connection with FIG. 7 refers to a region including the quadriceps tendon but does not include the patella, and the term the region below the patella refers to a region including the patellar tendon but not including the patella. Since a patient may be standing, sitting or lying down, the term above and below do not necessarily correlate to the direction of gravity. Scan path 70 includes a second path 74 extends over and/or proximate to the region of the patellar tendon. Scan path 70 includes a third path 76 that extends over or proximate to the femoral cartilage and a fourth path 78 that extends over and/or proximate the posterior cruciate ligament. However, other paths covering various anatomical aspects of the knee joint are also contemplated. While the ending point of scan path 72 may also be the beginning of scan path 74 it is also contemplated that the ending of each scan path does not correlate with the beginning point of each subsequent scan path.

In one implementation the robotic arm 46 is a spherical robot as is known in the art employing a spherical coordinate system. In one implantation the three dimensional reference of the points identified of the patient's knee are based on a spherical coordinate system. The three dimensional mapping is correlated to a known three dimensional position relative to the robotic system 16. In one implementation other reference frames may be used to identify the location of a point on a patient's knee relative to the robotic system 16. For example the location of various points on a patient's knee may be relative to a location on an interface portion 48 of ultrasound probe 18 as will be described in further detail herein below. The ultrasound probe interface portion 48 is the portion of the ultrasound probe which is pressed against a patient's skin during an ultrasound procedure.

Referring to FIG. 5 the robotic system 16 includes a robotic arm 46 having a plurality of links 50 operatively connected together by joints 52 allowing one or both of a rotational motion and translational displacement. The links 50 of the robotic arm 46 can be considered to form a chain with the free end of the chain of links having an end effector 54 operatively securing ultrasound probe 18. In one implementation robotic arm 46 has a known location relative to patient support 12. The robotic arm 46 has multiple degrees of freedom sufficient to obtain ultrasound images about a patient's knee.

Referring to FIG. 1 the robotic system includes a robotic control module 56 through a processor using instructions provided therein or in memory that calculates the position of the ultrasound probe interface portion 48 of the robotic arm relative to the outer portions of the patient's knee. In one embodiment the ultrasound probe 18 is secured to a portion of robotic arm 46. The control module provides commands to the robotic arm 46 to move such that the interface 48 of ultrasound probe 18 is adjacent to an outer portion of patient's knee and along a scanning path. Stated another way, the robotic arm 46 is moved such that the interface 48 of ultrasound probe 18 contacts a plurality of locations on an outer portion 46 of the patient's knee along a scan path 70. In one implementation the interface surface 48 of ultrasound probe 18 is positioned adjacent the outer portion 36 of the patient's knee with sufficient force, orientation and manner to obtain an ultrasound scan of the anatomical portions of the patient's knee that are covered by the patient's skin. In the example orientation of the ultrasound probe 18, it includes the angle of a longitudinal axis of the ultrasound probe 18 relative to a normal vector of the point of contact to the outer portion 36 of the patient's leg.

The Scan path 70 as described herein below includes a path that the ultrasound probe 18 travels to obtain an ultrasound scan of a patient's knee. In one implementation the scan path 70 is a linear or non-linear pathway that the ultrasound probe 18 contacts from a first point to a second point on the scan path 70 and all points in between. The Scan path 70 may provide a number of paths in which the ultrasound probe travels. In one implementation there are a number of discrete scan paths that the ultrasound probe travels in order to obtain sufficient ultrasound images of the patient's knee. In another implementation there is a single scan path 70 that allows the ultrasound probe to navigate about the outer portion of the patient's knee in order obtain sufficient ultrasound image data to analyze the patient's knee. In one implementation there is different scan path for different orientations of a patient's leg and knee. For example in one orientation a patient's leg is straight and in a second orientation a patient's leg is bent at the knee joint. In a third orientation a patient's leg is twisted about the knee join. Stated another way in one orientation the patient's leg including the femur is adjacent to a portion of the bed 24 and a portion of the patient's tibia is adjacent extension portion 26 when the axis of the bed 24 is collinear with the axis of extension portion 26. In a second orientation the patient's leg including the femur 66 is adjacent to a portion of the bed 24 and a portion of the patient's leg including the tibia 68 is adjacent to a portion of the extension portion when the bed axis and extension axis are not collinear. In the third orientation the patient's leg including the femur is adjacent to a portion of the bed 24 and a portion of the patient's leg including the tibia is adjacent a portion of the extension portion when the bed axis and extension axis are rotated relative to one another resulting in rotation of the femur relative to the tibia about the knee joint. It is also contemplated that the femur and tibia may both be rotated relative to one another as well as not collinear.

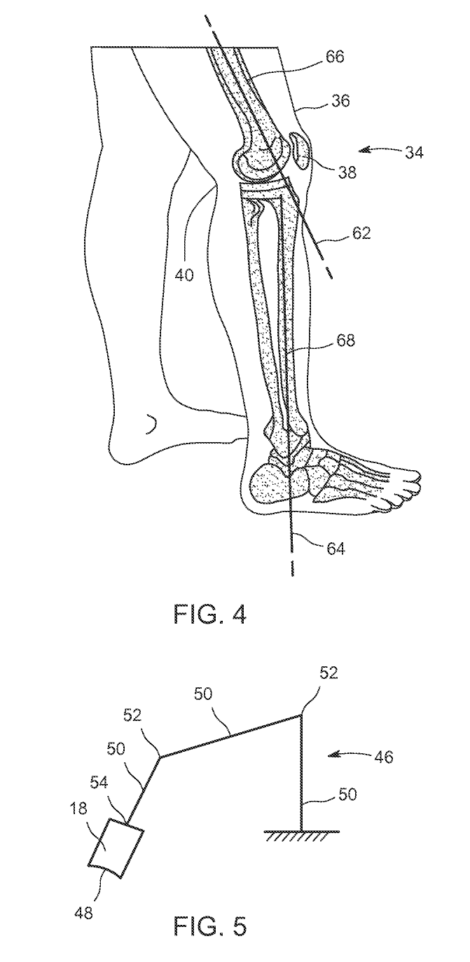

Referring to FIG. 4, an upper leg longitudinal axis 62 and a lower leg longitudinal axis 64 are collinear when the leg is straight and the knee is in a non bent orientation. The upper leg longitudinal axis 62 and lower longitudinal axis 64 are at an angle other than 0 degrees or 180 degrees when the knee is in a bent orientation. The upper leg portion includes the femur 66 and the lower leg portion includes the tibia 68.

The ultrasound system 20 includes an ultrasound probe 18 having a transducer, and an ultrasound processing unit 46. In one implementation the ultrasound system includes a display and an input device. A transmitter and a receiver are operatively connected to the transducer to transmit data between the transducer 44 and the processing unit 46. According to one implementation the ultrasound processing unit 46 follows instructions contained in a memory and receives ultrasound echo signals from the ultrasound transducer 44 and analyzes such signals, wherein the results of such analysis are presented on display 48 or stored for analysis.

The Ultrasound data obtained is used by a physician or operator to diagnose an arthritis condition in the patient's knee joint. In one implementation B-mode data and Doppler data from an initial ultrasound scan or subsequent ultrasound scan of the patient's knee is used in the algorithm to modify the position of the knee joint by either changing the angle of bend in the patient's knee and/or rotating a lower portion of the patient's leg relative to an upper portion of the patient's leg to apply a rotational element to the patient's knee for additional ultrasound scanning.

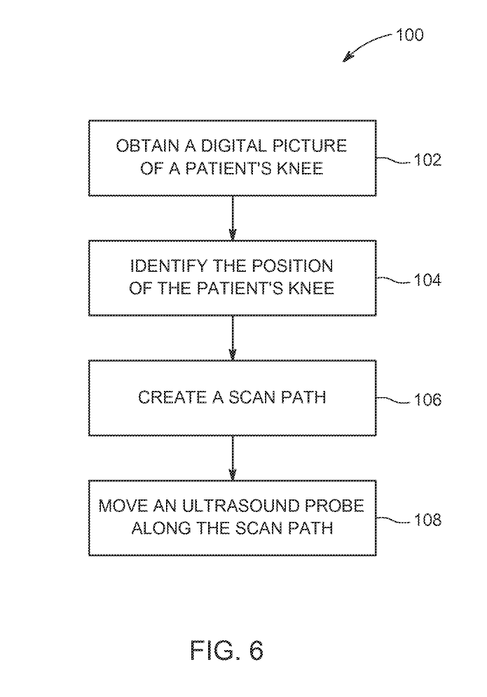

FIG. 6 is a flow diagram of a method 100 for automatically scanning a knee joint with ultrasound. In one implementation, method 100 may be carried out by the automated ultrasound system 10. In another implementation, method 100 may be carried out by other ultrasound imaging systems.

Referring to FIG. 6 as indicated by block 102 method 100 of an automated robotic ultrasound scan of a knee includes obtaining a digital picture of the patient's knee. In one implementation the digital picture is obtained with at least one digital camera. In one implementation it is also contemplated to obtain a digital picture of the patient's knee with other sensing devices as discussed herein above. The digital picture of the patient's knee is the image of the outer portion 36 of the patient's knee and may include but not limited to the region of the knee proximate to the patella, the region of the popliteal fossa and the outer portion of the knee there between. In one embodiment the digital picture of the patient's knee includes the outer portion 36 circumferentially about the patient's knee such that a digital picture of the entire outer periphery of the patient's leg in the region of the patient's knee is obtained.

As indicated by block 104, method 100 includes applying instructions to a processor to identify the position of the patient's knee relative to a known datum based on an algorithm and the digital pictures. In one implementation the processor creates a three dimensional model of the outer portion 36 of the patient's leg adjacent the patient's knee. The position and dimensions of the outer portion 36 of the patient's leg is determined and included in the model.

As indicated by block 106, method 100 includes creating a scan path 70 about the patient's knee based on the position of the patient's knee. In one implementation the scan path 70 is determined relative to a datum and the dimensions of the outer portion of the patient's leg;

As indicated by block 108, method 100 includes providing instructions to a robotic processor to robotically move an ultrasound probe along the scan path 70.

Once the scan path 70 is determined, and the ultrasound probe is moved along the scan path 70, the method further includes obtaining ultrasound images from the ultrasound probe. The ultrasound images are transferred to an imaging processor to analyze the ultrasound images for arthritis diagnosis.

In one implementation, the patient support receives robotic instructions to robotically and automatically adjust the patient support and/or extension region to reposition the position the patient's knee in a given orientation. The method 100 may then be repeated to obtain new ultrasound data based on the adjusted orientation of the patient's knee.

In one implementation the adjustment of the patient's knee to a different orientation is determined by an algorithm that is a function of the ultrasound data obtained during a first ultras sound scan along a first scan path 70. Based on a first analysis of the presence of arthritis in one specific area of the patient's knee based on the ultrasound data, an algorithm will direct movement of the joint to obtain additional ultrasound data of the knee in a second orientation to supplement the diagnosis of the arthritis.

In one implementation the scan path 70 is updated and revised during movement of the ultrasound probe and acquisition of ultrasound data if the position of the patient's leg has deviated from a predetermined limit from a first location and/or orientation.

In a further implementation the method of updating the scan path 70 with an algorithm is a function of the digital camera image.

In one implementation a doctor or medical operator identifies a region of interest of a patient's leg through a user interface of the system. An algorithm then calculates the scan path 70 to obtain the ultrasound image of the region of interest identified by the medical operator. The medical operator may identify the region of interest by using an interface such as a mouse or other computer input such as touch screen and identify the region of interest on the image of the outer portion of the patient's leg. In one implementation a medical operator may identify a predetermined anatomical region and the algorithm automatically calculates a scan path 70 to obtain an ultrasound image of the predetermined anatomical region of interest based on the graphical model of the outer portion of the patient's leg.

By way of a non-limiting examples, the medical operator may elect cartilage between the femur and tibia as an anatomical structure of interest

While the preferred embodiments of the invention have been illustrated and described, it will be appreciated that various changes can be made therein without departing from the spirit and scope of the invention. For example, although different example embodiments may have been described as including one or more features providing one or more benefits, it is contemplated that the described features may be interchanged with one another or alternatively be combined with one another in the described example embodiments or in other alternative embodiments. One of skill in the art will understand that the invention may also be practiced without many of the details described above. Accordingly, it will be intended to include all such alternatives, modifications and variations set forth within the spirit and scope of the appended claims. Further, some well-known structures or functions may not be shown or described in detail because such structures or functions would be known to one skilled in the art. Unless a term is specifically and overtly defined in this specification, the terminology used in the present specification is intended to be interpreted in its broadest reasonable manner, even though it may be used conjunction with the description of certain specific embodiments of the present invention.

What is claimed is:

1. An apparatus comprising:
a digital camera providing a digital picture of a patient's knee;
a processor receiving instructions to identify the position of the patient's knee from the digital picture;
the processor creating a scan path about the patient's knee;
a robotic arm supporting an ultrasound probe and receiving instructions from the processor to move the ultrasound probe along the scan path.

2. The apparatus of claim 1 including a positioning system providing instructions to a patient support to robotically position the patient's knee in a given orientation.

3. The apparatus of claim 2, wherein the positioning system moves the patient's knee from a first orientation to a second different orientation.

4. The apparatus of claim 2, wherein the first orientation is a straight orientation and the second orientation is a bent orientation.

5. The apparatus of claim 2, wherein the positioning system rotates the patient's leg about a longitudinal axis of a lower portion of the patient's leg.

6. The apparatus of claim 1, wherein the scan path extends substantially about an outer portion of patient's knee including a patella region and a popliteal foss region of the patient's knee.

7. The apparatus of claim 1 including an input device for a medical operator to identify the region of interest of the patient's knee joint for ultrasound imaging.

8. The apparatus of claim 1, wherein the scan path is updated during movement of the ultrasound probe and acquisition of ultrasound data.

9. The apparatus of claim 8, wherein the scan path is updated as a function of a subsequent digital picture.

10. The apparatus of claim 8, wherein the scan path is updated by an algorithm as a function of the ultrasound image data.

11. A method comprising:
obtaining a digital picture of a patient's knee with a digital camera;
applying instructions to the processor to identify the position of the patient's knee relative to a known datum based on an algorithm and the digital picture;
creating a scan path about the patient's knee based on the position of the patient's knee; and
providing instructions from a robotic processor to robotically move an ultrasound probe along the scan path.

12. The method of claim 11, further including obtaining ultrasound image from the ultrasound probe.

13. The method of claim 11, further including providing instructions to a positioning system to robotically position the patient's knee in a given orientation.

14. The method of claim 13, further providing instructions to the positioning system to move the patient's knee from a first orientation to a second different orientation.

15. The method of claim 11, further providing instructions to the positioning system to rotate the patient's leg about a longitudinal axis of a lower portion of the patient's leg.

16. The method of claim 11, further creating a scan path extending substantially about an outer portion patient's knee including a patella region and a popliteal foss region of the patient's knee.

17. The method of claim 11 wherein creating a scan path is a function of a region of anatomical interest identified through a user input by a medical operator.

18. The method of claim 11, further updating the scan path during movement of the ultrasound probe and acquisition of ultrasound data.

19. The method of claim 11, further updating the scan path with an algorithm as a function of the digital camera image.

20. The method of claim 17, further updating the scan path with an algorithm as a function of the ultrasound data.
